(19) United States
(12) Patent Application Publication
(10) Pub. No.: US 2014/0371189 A1
(43) Pub. Date: Dec. 18, 2014

(54) TESTOSTERONE REPLACEMENT THERAPY IN ANIMALS, INCLUDING DOGS

(71) Applicant: David Bieber Consulting, LLC, Weston, FL (US)

(72) Inventor: David Bieber, Weston, FL (US)

(73) Assignee: DAVID BIEBER CONSULTING, LLC, Weston, FL (US)

(21) Appl. No.: 14/188,266

(22) Filed: Feb. 24, 2014

Related U.S. Application Data
(60) Provisional application No. 61/834,295, filed on Jun. 12, 2013.

(51) Int. Cl. A61K 31/568

(52) U.S. Cl. CPC A61K 31/568
USPC 514/178

(57) ABSTRACT

A method of treating an animal which does not naturally produce sufficient quantities of testosterone includes periodically administering a testosterone to the animal. The method includes conducting an initial examination of the animal; determining a testosterone level of the animal; administering a testosterone to the animal; determining the testosterone level of the animal to which the testosterone has been administered; and periodically administering the testosterone to the animal and monitoring the testosterone level, based on the detected testosterone levels and clinical signs.

### BACKGROUND OF THE INVENTION

1. Field of Invention

Debilitated dogs that have arthritis or spinal disease or are simply old sometimes have difficulty getting up from a laying position or walking. Such debilitation can result from a deficiency in the hormone testosterone. Testosterone maintains muscle tone and bone density in mammals, including dogs and horses. Neutered and spayed dogs and horses have zero to minimal amounts of testosterone in their bodies, and can lack muscle tone and bone density and therefore sometimes cannot get up or walk properly.

2. Description of the Related Art

It has been previously known to administer testosterone to humans for various clinical reasons. Additionally, it has been previously known to administer testosterone to some non-human animals, such as dogs and horses; however, it has not been previously known to do so for treating osteoarthritis (also known as degenerative joint disease), intervertebral disc disease, hip dysplasia, weakness due to old age, muscle atrophy and joint pain in non-human animals.

### SUMMARY OF THE INVENTION

It is an object of the invention to provide a method for the administration of testosterone to a non-human animal in need thereof.

It is a further object of the invention to provide therapy for testosterone deficient animals to increase their testosterone levels and to provide associated benefits with regards to mobility and energy levels.

This and other objects are achieved using the invention as herein described.

These and other objects of the invention, as well as many of the intended advantages thereof, will become more readily apparent when reference is made to the following description taken in conjunction with the accompanying drawing.

### DETAILED DESCRIPTION OF THE PREFERRED EMBODIMENTS

Until the present invention, testosterone replacement therapy had not been used for animals generally, and specifically not for dogs and horses. In most instances, the therapy of the present invention will be employed when the animal no longer has a naturally occurring source of testosterone, for example in a neutered or spayed animal. However, low testosterone may be caused by other conditions, such as old age.

In testosterone therapy according to the invention, testosterone can be administered into the animal's body in a number of ways. The most common method is intramuscular (IM) injection with a syringe. Other delivery methods include (i) transdermal application through a gel, cream, or patch applied to the skin, (ii) orally by swallowing tablets (this method is uncommon as it has been shown to have negative effects on the liver), (iii) sublingually/buccally by dissolving a tablet under the tongue or against the gums; or by a pellet inserted under the skin. The testosterone-delivery method used will depend on the type of medication available in the country of treatment, the health risks/benefits for the canine patient and cost.

Testosterone is not stored by the body for future use. Therefore, testosterone must be administered in time intervals in appropriate dosages. In a preferred embodiment of the present invention, on average once-monthly injections are used so in order to maintain healthy testosterone levels. Injectable and subcutaneous testosterone pellets remain active in the body the longest. Injectable testosterone is typically administered once every three to six weeks, based on the animal's clinical signs and the specific type of injectable testosterone. Subcutaneous testosterone pellets are normally replaced every 3-6 months. Transdermal testosterone (patch, gel, or cream) is typically applied to the skin in smaller daily doses; oral and sublingual/buccal testosterone is also typically taken daily.

### “Normal” Testosterone Levels

Testosterone levels are usually confirmed through a blood test called a “serum total testosterone test.” Testosterone exists in the bloodstream in two forms—“bound” testosterone and “free” testosterone. The majority of bound testosterone in the body is chemically bound to a protein called “sex hormone binding globulin” (SHBG). The remaining bound testosterone in the system is mostly bound to albumin, another protein. Free testosterone is not chemically attached to any proteins and is considered the “active” form of testosterone, as it is readily available to bind to androgen receptor sites on cells.

A serum total testosterone test measures the total of bound and free testosterone in the system. The normal test level of combined bound and free testosterone in male human bodies can range anywhere from 300-1100 ng/dl (nanograms per deciliter). For dogs, however, normal total testosterone levels in intact males will usually be greater than 1000 pg/ml (picograms per milliliter), while normal total testosterone levels in intact females will usually be about 150 pg/ml. Neutered or spayed dogs usually have total testosterone levels of 0-40 pg/ml. It should be noted that although the disclosed method could be performed based on either free testosterone or total testosterone, there are currently no commercially available assays for free testosterone in animals. As such, total testosterone alone can be relied upon.

It is useful to also separately measure the level of free testosterone in the system, as this may be more indicative of how well hormone therapy is progressing. Levels of free testosterone can range between 0.3%-5% of the total testosterone count, with about 2% considered to be an optimal level. In general it is recommended that both total and free testosterone levels be checked.

In general, the testosterone therapy of the present invention is most effective with consistent monitoring (i.e. every 3-6 months). Small adjustments in dosage (either increases or decreases) can make a significant difference in testosterone levels.

### Testosterone Esters

Much of the testosterone that is prescribed for the purposes of hormone therapy is in the form of testosterone “esters.” An ester is simply a name for a chemical compound that is formed from reaction between a carboxylic acid and an alcohol. A simple chemical diagram of this reaction is as follows:

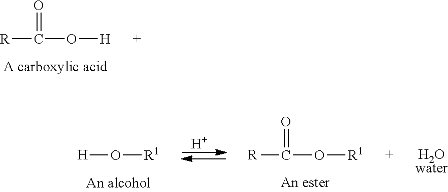

(where R and R¹ are general hydrocarbon groups)

The chemical structure of free testosterone (chemical formula C₁₉H₂₈O₂), and of two different esters of testosterone, testosterone cypionate, and testosterone enanthate, is as follows:

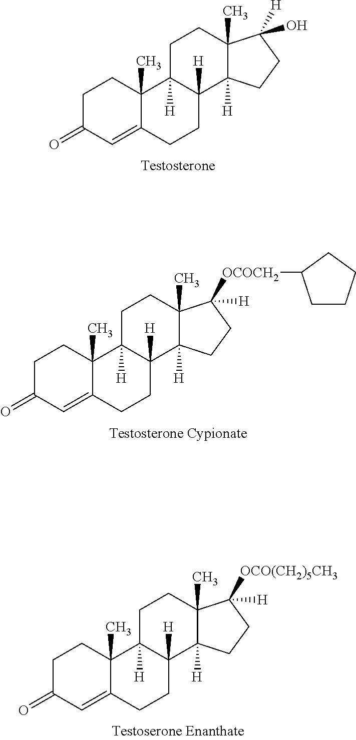

There are a number of different esters of testosterone, including the commonly prescribed injectables of testosterone enanthate and testosterone cypionate, as well others such as acetate, propionate, phenylpropionate, isocaproate, caproate, decanoate, and undecanoate. Each of these different esters is a molecular chain composed of carbon, hydrogen, and oxygen atoms. The main difference between the different esters is how many carbon and hydrogen atoms make up the chain. For example, the propionate ester is composed of 3 carbons, 6 hydrogens, and 2 oxygens, whereas the cypionate ester is composed of 8 carbons, 14 hydrogens, and 2 oxygens.

The advantage of using esterified testosterone is that esterified forms of testosterone improve the solubility of testosterone in oil, which in turn slows the release of the testosterone from the site where it enters the body.

Testosterone, in its free, non-esterified form, has poor solubility in both oil and water, although it can be suspended in water. A non-esterified testosterone is available in an aqueous injectable form, such as the product sold under the trademark AQUAVIRON.

However, this form of testosterone stays active in the body for a very short period of time (only a matter of hours). Because of this, it must be injected on a daily basis in order to maintain a continuous level of testosterone in the blood. Therefore it is rarely used for testosterone replacement therapy as an injectable.

Esterified testosterone is less soluble in water than free testosterone, but is more soluble in oil. Moreover, as a general rule, the more carbon atoms in the ester radical, the more soluble the ester is in oil. For example, testosterone propionate (with 3 carbon atoms in the ester group) is less soluble in oil than testosterone cypionate (with 8 carbon atoms in the ester group).

The term for the ratio between oil and water solubility of a testosterone ester is called the “partition coefficient”:—the higher the solubility in oil, the higher the partition coefficient.

The partition coefficient of the ester is important because it affects how long the testosterone will remain in the system. If the testosterone transfers too quickly from the oil to the patient's blood, the testosterone level spikes rapidly and then quickly drops once the dose has been used up. If free testosterone injected into the muscle from a water suspension (as in Aquiviron), the testosterone is essentially immediately available to the bloodstream due to its low partition coefficient, and thus there is an immediate spike of testosterone which is used up quickly in the body.

Testosterone cypionate, by contrast, has a high partition coefficient. When injected into the muscle, the drug remains in its esterified form in a deposit in the muscle tissue. From there, it slowly enters the circulatory system as it is picked up in small quantities by the blood. Once the esterified testosterone is in the blood stream, “esterase enzymes” cleave off the ester chain in a process known as “hydrolization,” leaving the testosterone in its free form to perform its various actions and effects.

### Testosterone Delivery Methods

### Injectable Testosterone

The dosage amount and timing for injectable testosterone will depend largely upon which ester is being used, as well as the subject's response to the hormone. In general, human dosages will vary between 50 mg and 300 mg per injection, depending on the ester and the dosing regimen. An average injectable dose for humans is about 200-250 mg every two weeks, though many humans inject 100 mg about every week, or other variations depending on their own bodies' needs and sensitivities. For animals, this injection regimen is modified to reflect the weight and clinical signs of the animal in question.

There are a number of different types of injectable testosterone; availability may vary by country. The drug names for the same testosterone ester may also differ depending on the company who produces it. The following is not an exhaustive list, but does cover the most common forms of injectable testosterone. However, any brand or form of injectable testosterone, may be used in the present invention. Additionally, as discussed below, non-injectable forms of testosterone, such as pellets, gels, creams and the like, can be utilized.

### Injectable Esters for Testosterone Therapy

Testosterone Enanthate: Chemical Formula C₂₆H₄₀O₃

Testosterone enanthate is one of the main forms of testosterone prescribed to men in the United States. It is a slow-acting ester with a release time between 8-10 days. It is available in the United States under the trademark DELATESTRYL which is suspended in sesame oil. Testosterone enanthate is typically injected anywhere between once every week to once every three weeks. Generic testosterone enanthate can also be obtained through a compounding pharmacy; such pharmacies can mix T-enanthate in sesame oil, cottonseed oil, or any other appropriate oil.

Testosterone Cypionate: Chemical Formula C₂₇H₄₀O₃

Testosterone cypionate is the other principal injectable form of testosterone prescribed to men in the United States. It is a slow-acting ester with a release time between 8-10 days, similar to that of testosterone enanthate. It is available in the United States under the brand DEPO-TESTOSTERONE, in which the testosterone cypionate is suspended in cottonseed oil. Testosterone cypionate is typically injected in humans anywhere between once every week to once every three weeks. For dogs, the period between injections is typically every 3 to 6 weeks. Generic testosterone cypionate can also be obtained through a compounding pharmacy; such pharmacies can mix testosterone-cypionate in sesame oil, cotton seed, or any other appropriate oil.

Testosterone Propionate: Chemical Formula C₂₂H₃₂O₃

Testosterone propionate is a fast-acting testosterone ester with a release time of 3-4 days. In order to keep blood levels of testosterone from excessive fluctuation, testosterone propionate is usually injected between one to three times a week. Brand names of testosterone propionate include “Testovis” and “Virormone.”

Testosterone Phenylpropionate: Chemical Formula C₂₈H₃₆O₃

Testosterone phenylpropionate is a slow-acting testosterone ester, with a release time of 1-3 weeks. A popular name brand for testosterone-phenylpropionate is TESTOLENT™.

SUSTANON®

SUSTANON® is the brand name for two forms of injectable testosterone that contain a blend of testosterone esters. SUSTANON® 100 contains three testosterone esters: testosterone propionate, testosterone phenylpropionate, and testosterone isocaproate (C₂₅H₃₃O₃) SUSTANON® 250 contains four testosterone esters: testosterone propionate, testosterone phenylpropionate, testosterone isocaproate, and testosterone decanoate (C₂₉H₄₆O₃). Each formula features fast-acting and slow-acting esters, and can be injected anywhere from once every week to once every four weeks.

OMNADREN™

OMNADREN™ is the brand name for a blend of four testosterone esters: testosterone propionate, testosterone phenylpropionate, testosterone isocaproate, and testosterone decanoate. In the past, OMNADREN™ consisted of a blend of different esters, but now is essentially the same formula as SUSTANON®, mentioned above. OMNADREN™ features both fast-acting and slow-acting esters, and can be injected anywhere from once every week to once every four weeks.

### Aqueous Testosterone Suspension

In the United States, injectable aqueous (non-esterified) testosterone is available, but it is very short-acting; it is completely released in the system within a matter of hours. Therefore, it is not typically used for men in transition, as it would require constant re-injection to maintain regular blood levels. The brand name for aqueous testosterone suspension is AQUAVIRON.

### Transdermal Testosterone

Transdermal testosterone is delivered topically, through the skin, by the use of a patch, gel, or cream. Transdermal testosterone is usually applied to the skin daily in small doses in an effort to keep a steady level of testosterone in the system at all times. Transdermal delivery of testosterone avoids the peaks and valleys in testosterone-levels sometimes associated with injectable testosterone. With injectable systems, testosterone levels can reach a low-point a few days before the next shot is due, which may lower the effectiveness of the therapy in that time period. Daily transdermal application of testosterone can help alleviate such problems. For transdermal delivery of testosterone to animals, including dogs, the animal's fur may need to be shaved for the application to be most effective. Transdermal application is also effective for those animals who are not comfortable with needles and injections.

### Testosterone Patches

There are currently two brand-name testosterone patches available in the United States for human use: ANDRODERM® and “TESTODERM™. Both ANDRODERM™ and “TESTODERM™ are very fast-acting once they have permeated the skin. The testosterone in the patches is suspended in an alcohol-based gel in order to deliver testosterone efficiently into the body, chemical enhancers are added to the patch to increase permeability of the skin.

### Testosterone Gel and Cream

There are currently two brand-name versions of testosterone gel available: ANDROGEL and TESTIM. There are no brand-name testosterone creams at this time. Both cream and gel formulations of testosterone can be made by compounding pharmacies. Gel formulations of testosterone are typically alcohol-based, whereas creams are typically safflower oil-based. The testosterone in creams and gels is typically very fast-acting once absorbed through the skin. Thus, it must be applied once or twice daily to maintain appropriate testosterone levels. Creams and gels are applied directly onto the skin. Transfer of the testosterone from the site can be prevented by keeping the area covered.

ANDROGEL

ANDROGEL is a clear, alcohol-based gel which contains 1% non-esterified testosterone. It is very fast-acting once it has been absorbed by the skin, and therefore must be applied 1-2 times daily to maintain testosterone levels. It is available in either unit-dose packets or multiple-dose pumps. The unit dose packets contain either 25 mg or 50 mg of testosterone. Approximately 10% of the applied testosterone from the packets is absorbed into the system, resulting in an effective dose of 2.5 mg or 5.0 mg per packet, respectively. These dosages need to be normalized for the animal employing the therapy, as the above dosages are for humans. Androgel should be applied to clean, dry skin and should not be applied to the genital area. Application sites should be allowed to dry for a few minutes prior to dressing.

TESTIM

Testim is also a clear, alcohol-based gel that contains 1% non-esterified testosterone. Like ANDROGEL it is very fast acting once it has been absorbed by the skin, and so must be applied 1-2 times daily to maintain testosterone levels. It is available in 5.0 g unit-dose tubes. A 5.0 g unit dose tube contains 50 mg of testosterone. Approximately 10% of the applied testosterone from the tube is absorbed into the system, resulting in an effective dose of 5.0 mg. TESTIM should be applied to clean, dry skin.

### Compounded Creams and Gels

Compounded creams and gels can be mixed by compounding pharmacies, and are similar in dosing, application, and precautions to what is described above for ANDROGEL and TESTIM. There are two advantages of using compounding pharmacies to make a testosterone gel or cream. The first advantage is cost, because a compounded gel will usually be cheaper than the brand name products The second advantage is customization, a veterinarian can write a prescription of varying concentration for gels or creams.

### Oral Testosterone

Chemically unbound testosterone, if taken orally, is not effective since it is immediately deactivated by the liver. However a chemically modified form of testosterone has been successfully shown to be effective when taken orally: testosterone undecanoate. Testosterone undecanoate is a safe oral form of testosterone designed to be absorbed through the small intestine into the lymphatic system, posing less burden on the liver. Brand names for testosterone undecanoate include ANDRIOL, ANDROXON, UNDERSTOR, RESTANDOL, and RESTINSOL.

One disadvantage of orally administered testosterone undecanoate is that it is eliminated from the body very quickly, usually in 3-4 hours. Thus, frequent administration is necessary—usually between 3-6 capsules a day. This can prove to be expensive when compared to injectable testosterone. These dosages would need to be normalized for the animal employing the therapy, as the above dosages are for humans.

### Sublingual/Buccal Testosterone

Sublingual and buccal testosterone delivery entails either placing a dissolving tablet under the tongue (sublingual) or by placing a tablet against the surface of the gums (buccal). It is different from oral delivery in that very little of the substance is swallowed, avoiding potential liver toxicity. This may be difficult for an animal to do, but it is considered as a possible way to employ this therapy.

### Sublingual Testosterone

Sublingual testosterone can be obtained through compounding pharmacies.

### Buccal Testosterone

In 2003, the FDA approved a sustained-release buccal testosterone tablet called STRIANT. It acts by adhering to the buccal mucosa (the small depression in the mouth where the gum meets the upper lip above the incisor teeth). Once applied. the tablet softens and delivers testosterone through the buccal mucosa, where it is then absorbed directly into the bloodstream, bypassing the gastrointestinal system and liver. The recommended human dosage for STRIANT is to replace the tablet about every 12 hours.

### Subcutaneous Testosterone Pellet

Another relatively new form of testosterone delivery is via a pellet of pure, crystalline testosterone implanted beneath the skin. The pellets are about the size of a grain of rice, and are typically placed in the buttocks or abdomen. The insertion of the pellets is a quick procedure, usually done under local anesthesia. Pellets are typically replaced after 3-4 months. TESTOPEL is a brand name for testosterone pellets in the United States. A 200 mg testosterone pellet releases testosterone at a steady rate of 1-3 mg per day. Several pellets can be inserted at the same time to increase dosage.

### Protocol for Administration of Testosterone to Dogs, Horses, or Other Animals

In order to treat dogs, horses, or other animals in need of testosterone replacement therapy, the following protocol should be followed:
(1) Obtain information about potential patient via a clinical intake process and questionnaire such as the example shown in the drawing figure;
(2) Conduct a general physical examination on potential patient;
(3) Conduct laboratory tests on potential patient's blood and/or urine, including measuring initial testosterone levels in the patient's blood. This can be total testosterone or free testosterone. If anything of these tests produce abnormal results, these should be corrected before starting testosterone replacement therapy;
(4) Provide first dose of testosterone (preferably by injection);
(5) Provide a second dose of testosterone (typically, but not necessarily, about one month after step (4));
(6) Check testosterone blood level (about one week after step (5));
(7) Provide a third dose of testosterone (about one month after step (4));
(8) Provide additional doses of testosterone at 3-6 week intervals, depending on testosterone levels and clinical signs observed by the veterinarian and the animal's owner.

### Client Intake

In the client intake process, it is important to obtain information about the patient's clinical history, such as: (1) how old was your animal when you acquired him/her? (2) at what age was the animal neutered/spayed? (3) pertinent medical history including accidents or illnesses, surgeries, previous radiographs (such as MRI/CAT); (4) at what age did the animal become symptomatic with his/her current problem? (5) current symptoms; (6) current diet; (7) animal's typically daily activities; (8) animal's recovery time from exercise.

Based on the information obtained during the intake process, the veterinarian can make a determination of whether the animal is a good candidate for the testosterone replacement therapy.

### Medical Examination

The animal will be given a typically physical examination, which can include general exam, neurological exam, rectal exam, and radiographs, or MRI's. This is to assess the general well-being of the animal, and again will inform the veterinarian as to the animal's suitability for testosterone replacement therapy.

### Laboratory Tests

In general, the laboratory tests which will be run include a complete full panel chemistry blood test, a CBC with differential, a thyroid blood test, a urinalysis, and total testosterone blood level. If anything on the laboratory tests is elevated or decreased, these cases should be treated and re-tested, and be considered before testosterone therapy is started.

If the patient's laboratory results and exam are normal and/or treated, such as, liver disease, kidney disease, urinary tract infections, thyroid disease, etc., then the patient can be started on the testosterone therapy.

### Testosterone Doses

In general, according to the present invention, any of the aforementioned forms of testosterone can be administered to the animal. However, according to the invention, preferably the testosterone is given by intramuscular injection, and preferably the testosterone is in the form of testosterone cypionate.

When the administered testosterone is testosterone cypionate, the preferred dosage is from about 0.5 to about 3.0 mg per kg (of dog's weight) for neutered medium and large male dogs (i.e., typically a male dog having a weight of from about 25 to about 150 plus pounds), and from about 0.25 to about 1.5 mg per kg for neutered small breed male dogs (i.e., typically a male dog having a weight of from about 2 to about 24 pounds). According to an even more preferred embodiment of the invention, when the administered testosterone is testosterone cypionate, the preferred dosage is about 2 mg per kg for neutered medium and large male dogs, and about 1 mg per kg for neutered small breed male dogs. Female spayed dogs will be typically be given about 15% of the above-described dosages.

### Long-Term Monitoring

In general, once the animal's testosterone level has been brought to the desired level, monitoring of the animal's health and levels must be undertaken. Typically, total testosterone blood levels are measured every six months, allowing for adjustments in dosage amounts if required. General chemistry and CBC with differential are conducted after three months, and then every six months thereafter. Finally, thyroid blood levels and urinalysis are normally performed every 12 months.

### EXAMPLES

### Example 1

A study was undertaken to determine if dogs compromised by osteoarthritis (also known as degenerative joint disease), intervertebral disc disease, hip dysplasia, weakness due to old age, muscle atrophy and joint pain experienced improvements following testosterone replacement therapy. Twenty-seven dogs were treated with testosterone as outlined above, and their medical records were reviewed. Data from the medical records were analyzed to determine the efficacy of the treatment protocol. Data recorded from medical records included breed, date of birth, age, sex, neutered vs. intact, baseline testosterone level, date of testosterone injections and improvement status (improved vs. no noticeable improvement). Data analyses included frequencies and descriptives. All analyses were conducted using SPSS 14.0. Improvement for each dog was defined as an increase in mobility, and/or an increase in energy, and/or a decrease in pain medication, and/or a reduction in muscle atrophy. Two dogs were excluded from the analysis due to missing data.

Following informed assent by each dog owner, each dog underwent an initial clinical exam including a detailed health history, list of medications, a CBC, urinalysis and baseline testosterone level were conducted. Once the results of these tests were reviewed, participants were scheduled for an initial testosterone injection. Owners were called within two weeks of the initial injection for a brief follow-up to ascertain if there were any noticeable improvements or side effects. Patients were seen in the clinic for monthly follow-up visits after the initial injection. Changes in mobility, pain level/medications needed, energy and muscle tone were assessed and testosterone dosage was adjusted if needed prior to each injection. Follow-up testosterone levels were measured one week after the initial injection and on an as needed basis throughout the course of treatment.

The average age of participating dogs was 11.63 years (SD=2.67, range 5.5 to 18 years). Seventy-two percent n=18) of subjects were neutered males and 24 percent (n=6) of subjects were spayed females. One subject (4%) was an intact male. The participating dogs were from various breeds including 1 Australian Cattle Dog (4%), 1 Belgian Malinoisn (4%), 1 Boxer (4%), 1 Cocker Spaniel (4%), 1 Corgi (4%), 2 German Shepherds (8%), 2 Golden Retrievers (8%), 1 Great Dane (4%), 1 Hound (4%), 4 Labrador Retrievers (16%), 1 Pug (4%), 2 Rottweilers (8%), 1 Sheltie (4%), 1 Old English Sheepdog (4%), Spitz (4%), and 4 mixed breed dogs (16%).

The most common clinical issues among the participating dogs prior to the testosterone replacement therapy were the presence of arthritis, weakness in the limbs, incontinence, limping, difficulty getting up from lying down, ataxia, pain, hip dysplasia, decreased movement, knuckling, and atrophy. Testosterone levels at baseline ranged from less than 40 to 659.5. Nineteen of the 25 dogs (76%) had testosterone levels less than 40 at baseline, 1 subject (4%) had a testosterone level of 55.30, 1 subject (4%) had a testosterone level of 56, 1 subject (4%) had a testosterone level of 588.60 and 1 subject (4%) had a testosterone level of 659.50. Two dogs did not have an initial testosterone level conducted.

Multiple indications of improvement were seen during the course of treatment including: (1) being able to get up more easily, (2) becoming more mobile, (3) standing without assistance, (4) better weight bearing, (5) improved strength and stamina, and (6) increased jumping. Two dogs (8%) had to have their testosterone injection dosages lowered due to participants becoming too active.

Overall, ninety-two percent (n=23) of subjects made improvements after undergoing the study protocol. The two dogs which did not show improvement were the oldest patients in the study, and treatment was done as a last resort prior to making a decision regarding euthanasia.

Evaluation for side effects was conducted at monthly follow-ups. Throughout the course of this study, no side effects were observed for any of the dogs.

A majority of participants showed improvements in mobility, energy and pain level after undergoing a treatment protocol including testosterone injections. No side effects were reported during the data collection period.

### Example 2

A 28.7 pound, 12 year and two month-old neutered male Cocker Spaniel named Blackjack was evaluated because of his owner's concerns about declines in his level of mobility and arthritis. During the course of treatment, the patient suffered a distal humeral lateral condylar fracture of the left elbow. Blackjack completely healed from this fracture within two months of surgery and was able to walk two weeks post-surgery, while being treated with testosterone.

Blackjack's initial testosterone level was measured at less than 40 pg/ml during the initial screening process. (The normal level in intact male dogs is greater than 1000 pg/ml.) Blackjack's CBC showed results within normal limits and urinalysis results showed no infections, while. Alkphosphatase levels were slightly elevated (alk-phosphatase=138). During the second month of treatment, Blackjack's testosterone level was measured at 604.80 pg/ml and during the third month the testosterone level was measured at 1224.50 pg/ml.

During month one of the protocol, Blackjack was screened for treatment by conducting a thorough physical exam, complete blood chemistry, urinalysis, and testosterone level. The initial screening showed all lab values within normal limits with the exception of alk-phosphatase, which was slightly elevated (alk-phosphatase=138). The urinary analysis showed no infection. Blackjack's initial testosterone level was <40 pg/ml. An initial injection of testosterone IM was administered and Blackjack began treatment with Previcoxx 57 mg once per day. A follow-up was conducted by phone one day after the initial injection and again at two weeks post injection. No side effects were reported.

During month two, Blackjack's owner reported continued limping and diarrhea. Previcoxx was discontinued and Metacam was prescribed. During a follow-up appointment Blackjack received an injection of testosterone IM. A second testosterone level was run one week after the second injection which showed a testosterone level of 604.80 pg/ml. It was decided to increase the dosage of testosterone. At this stage, Blackjack had gained 1.1 pounds since beginning the treatment.

Blackjack was brought the clinic because of due to weight bearing lameness on the front left limb at the end of month two. Blackjack's owner reported that Blackjack had jumped off the table and had injured his leg. Radiographs were taken to rule out degenerative joint disease and neoplasia. During month three, the patient received another injection of testosterone IM. However, due to continued lameness, a consult with an orthopedic surgeon was scheduled, at which Blackjack was diagnosed with distal humeral lateral condylar fracture of the left elbow joint with failure of ossification. Blackjack was then operated on to treat the fracture.

During month 4, Blackjack's testosterone level was measured for a third time, and the results showed a testosterone level of 1224.50 pg/ml. Accordingly, the testosterone dosage was maintained. At this time a regular annual exam was conducted. Significant findings from this exam were slight periodontal disease, non-complete bilateral cataracts, and a fatty-type mass in the prepuce region. All other findings were within normal limits.

Monthly injections of testosterone continued. The dosage of testosterone had to be lowered after surgery due to concerns about Blackjack being too active after surgery to repair his fracture and was raised again after healing was complete. During month nine, Blackjack underwent further surgery to remove multiple skin growths.

During month ten Blackjack received an injection of testosterone, returning him to the dosage he received prior to his injury and subsequent surgery. During month eleven, the testosterone dosage was decreased due to patient mounting other dogs. During month twelve Blackjack continued receiving testosterone injections, and during month thirteen additional skin growths had to be removed.

Blackjack's owner was interviewed during week 57 of the protocol, and he reported improvements in Blackjack's ability to jump on furniture, ascend and descend stairs, and rise from sitting and lying positions. In addition, Blackjack walked faster, seemed less stiff, appeared to be in less pain, and had more energy since starting the protocol.

There appeared to be no adverse side effects from the treatment.

The invention being thus described, it will be apparent that the same may be varied in many ways. Such variations are not to be regarded as a departure from the spirit and scope of the invention, and all such modifications as would be recognized by one skilled in the art are intended to be included within the scope of the following claims.

What is claimed is:

1. A method of treating osteoarthritis, intervertebral disc disease, hip dysplasia, weakness due to old age, muscle atrophy and joint pain in a non-human animal, the method comprising:
(1) identifying a non-human animal in need of treatment of osteoarthritis, intervertebral disc disease, hip dysplasia, weakness due to old age, muscle atrophy and joint pain;
(2) determining a testosterone level of the non-human animal, the non-human animal lacking a natural source of testosterone;
(3) administering a testosterone to the non-human animal;
(4) determining the testosterone level of the non-human animal after step (3); and
(5) periodically administering the testosterone to the non-human animal and monitoring said testosterone level.

2. The method of claim 1, wherein following step (4), the non-human animal has a serum blood level of testosterone which approximates that of a similar non-human animal which produces testosterone naturally.

3. The method of claim 2, wherein the non-human animal is a dog.

4. The method of claim 2, wherein the non-human animal is a horse.

5. The method of claim 2, wherein the testosterone is administered by an injection.

6. The method of claim 5, wherein the testosterone which is injected is selected from the group consisting of testosterone enanthate, testosterone cypionate, testosterone propionate, testosterone phenylpropionate, testosterone isocaproate, testosterone cypionate and testosterone decanoate.

7. The method of claim 6, wherein the testosterone is testosterone cypionate.

8. The method of claim 7,
wherein when the non-human animal is a male, an amount of the testosterone cypionate that is administered is from about 0.5 to about 3.0 mg/kg of the weight of the non-human animal, and
wherein when the non-human animal is a female, an amount of the testosterone cypionate that is administered is from about 0.075 to about 0.45 mg/kg of the weight of the non-human animal.

9. The method of claim 8,
wherein when the non-human animal is a male, the amount of the testosterone cypionate that is administered is about 2 mg/kg of the weight of the non-human animal, and
wherein when the non-human animal is a female, the amount of the testosterone cypionate that is administered is about 0.3 mg/kg of the weight of the non-human animal.

10. The method of claim 7,
wherein when the non-human animal is a male, the amount of the testosterone cypionate that is administered is from about 0.25 to about 1.5 mg/kg of the weight of the non-human animal, and
wherein when the non-human animal is a female, the amount of the testosterone cypionate that is administered is from about 0.0375 to about 0.225 mg/kg of the weight of the non-human animal.

11. The method of claim 10,
wherein when the non-human animal is a male, the amount of the testosterone cypionate that is administered is about 1 mg/kg of the weight of the non-human animal, and
wherein when the non-human animal is a female, the amount of the testosterone cypionate that is administered is about 0.15 mg/kg of the weight of the non-human animal.

12. The method of claim 8, wherein the injection of the testosterone cypionate is administered once per month.

13. The method of claim 10, wherein the injection of the testosterone cypionate is administered once per month.

14. The method of claim 3, wherein the testosterone is administered by a method selected from the group consisting of injections, transdermal, patches, gels, creams, oral, sublingual, buccal, and subcutaneous pellet implantation.

15. The method according to claim 8, wherein the non-human animal is a neutered or spayed dog weighing about 25 pounds or more.

16. The method according to claim 10, wherein the non-human animal is a neutered or spayed dog weighing about 2 pounds to about 24 pounds.

17. A method of treating osteoarthritis, intervertebral disc disease, hip dysplasia, weakness due to old age, muscle atrophy and joint pain in a dog, the method comprising:
(1) identifying a dog in need of treatment of osteoarthritis, intervertebral disc disease, hip dysplasia, weakness due to old age, muscle atrophy and joint pain;
(2) determining a testosterone level of the dog, the dog lacking a natural source of testosterone;
(3) administering a first dose of testosterone by injection;
(4) administering a second dose of testosterone about one month after step (3) by injection;
(5) determining the testosterone blood level about one week after step (4);
(6) administering a third dose of testosterone about one month after step (3) by injection; and
(7) administering additional doses of testosterone at 3-6 week intervals by injection.

18. The method according to claim 17, wherein the dog is a spayed or neutered dog.

19. The method according to claim 17,
wherein the testosterone is testosterone cypionate, and
wherein when the dog is a male, an amount of the testosterone cypionate that is administered is from about 0.5 to about 3.0 mg/kg of the weight of the dog, and
wherein when the dog is a female, an amount of the testosterone cypionate that is administered is from about 0.075 to about 0.45 mg/kg of the weight of the dog.

20. The method according to claim 17,
wherein following step (5), in a male dog, the total testosterone level of the dog is greater than 1000 pg/ml, and
wherein following step (5), in a female dog, the total testosterone level of the dog is about 150 pg/ml.

21. The method according to claim 17, wherein, in step (2), the testosterone level of the dog is 0-40 pg/ml.
